(19) United States
(12) Patent Application Publication
(10) Pub. No.: US 2024/0185981 A1
(43) Pub. Date: Jun. 6, 2024

(54) Musculoskeletal Care Delivery Savings Strategy

(71) Applicant: Mark Meyocks, Yakima, WA (US)

(72) Inventor: Mark Meyocks, Yakima, WA (US)

(21) Appl. No.: 18/434,437

(22) Filed: Feb. 6, 2024

(51) Int. Cl. G16H 20/30; G16H 80/00

(52) U.S. Cl. CPC G16H 20/30; G16H 80/00

(57) ABSTRACT

Patent reduces musculoskeletal care delivery waste and costs, tangible and intangible, to plan sponsor and patient musculoskeletal from plan sponsor and/or network provider requiring a primary care visit for referral to physical therapy. Patent design removes requiring primary care appointments, via direct interactions with utility design reduces cost of delayed care and wasteful reimbursements in the current medical industrial complex scheme.

### CROSS REFERENCE TO RELATED APPLICATIONS

Not applicable

### STATEMENT REGARDING FEDERAL SPONSORED RESEARCH OR DEVELOPMENT

Not applicable

### BACKGROUND

### Musculoskeletal Care Delivery Savings Strategy (MCS)

The field of endeavor—Musculoskeletal Disease (pathology) and associated care delivery and costs (here after referred to as MSK).

The non-provisional patent design has been developed to provide plan eligible participants access to musculoskeletal care therapies quickly, without required primary care intervention for referral to physical therapist, a costly and ineffective expense, and provides incentive for self-remediation therapies to qualify for physical therapy referral and reimbursement of participants' physical therapy copays.

The non-provisional patent will disrupt the current medical industrial model of expensive and delaying musculoskeletal care delivery schemes. Patent design will provide tangible and intangible savings for musculoskeletal care delivery in the United States.

### Definitions Pertaining to Musculoskeletal Healthcare Delivery in the U.S.A.

Musculoskeletal System (MSK)—the muscles and joints of the human body.
Plan Sponsor—Employer, Multi-Employer (Taft-Hartley Trust), Healthcare Plan
Plan Design—Provides healthcare plan specifics regarding criteria of medical care delivery, responsibilities of eligible participants (copay, deductible, limits of care, family member, premium), options (constraints) for medical care providing physicians/clinics within or out the provided network of care. The forementioned are provided to participants in a Summary Plan Description (SPD) annually, by law.
Pathology—diseases of the human condition, may be acute (injury) or chronic (aging/wear and tear) related.
PMPM Fee—Per Member Per Month Fee (a negotiated fee for service paid to patent applicant).
Contracted (negotiated) Rate—Network provider paid to Primary for seeing participant.
Copay—Participant payment at time of visit or billed, as identified in the SPD provided annually to the participant or responsible party.
Administrator/Third Party Administrator (TPA)—Keeps track of benefit cashflow for participant accounting of pension, healthcare, retirement and holiday/vacation compensation and utilization throughout employment.

All benefits provided by plan sponsor (insurance premiums, deductible, co-pays, provider payments, etc.) are an administrator's responsibility.

The plan sponsors have options to self-administrate.

Have the network provider (Blue Cross, Aetna, CIGNA, etc.) administrate.

Hire an organization called a TPA.

Administrators reimburse providers, and advisors from the funds collected from participating employee compensation.
Network Provider (NP)—Insurance Company (Aetna, Blue Cross, etc.)

Provider of hospital group, surgical centers, primary and specialty care physicians as contracted.

Provides plan design for the plan sponsor structuring criteria for patient and provider (in and out of network care, billing rates, co-pays etc.).

Negotiates care fees with provided and adjudicates charges from participant care delivery.
Plan Sponsor (PS)—Employer, Health, and Welfare Trust (Taft-Hartley—Multi-employer Plan), Corporation, Other healthcare delivery sponsors.
Fiduciary/Decision Maker/Trustee (FD)—Decision makers that hire and monitor plan benefits and providers of healthcare.

Trustees of Taft-Hartley Funds are fiduciaries and held to high standard legally by Department of Labor regulations.

Not all decision makers are fiduciaries but maybe (i.e., plan attorney, plan consultants, administrator, etc.).
Eligible Participant (EP)—Individual participant covered under the healthcare plan.

Responsible for co-payments, deductible coverage limits, or costs as provided by the Network Provider/Plan Sponsor as defined annually in the plan design provided to the participant as a Summary Plan Description (SPD).
Primary Care Provider (DR.)—Physician, Physician's Assistant et. al. A general medical practitioner of healthcare services either in or out of the plan network. Can prescribe diagnostics, drugs, injections, and make referrals to other healthcare specialist providers.
Physical Therapy/Therapist (PT)—A trained specialist in identifying issues related to the musculoskeletal system (joints and muscles) of the participant.

Can through examination identify problems that may require more advanced care, (orthopedic interventions, surgery, etc.).

By license not allowed to prescribe diagnostics, drugs, injections, and may make referrals to other MSK specialist, however.
Orthopedic Provider (DO)—Network provider with specialized skills in specific MSK pathology (i.e., knee, wrist, spine, etc. as a surgeon or consultant),

Orthopedists provide intensive restorative care.

Orthopedists may use imaging to diagnosis MSK pathologies in EP.

Orthopedists are experienced healthcare professional providing advanced strategies to resolve pain and discomfort including:
Injections.
Pharmaceuticals.
Surgeries.

### Current MSK Delivery Scheme in the U.S.A. Overview

### Eligible Participant Has Musculoskeletal Pain and Discomfort

Eligible participants required to contact their primary care provider for an examination to determine the pathology of pain and discomfort, chronic/acute, arthritis/repetitive motion, other disease related eligible participant prior to physical therapist (specialist trained in examination and care of MSK system).
Current appointment scheduling 2-4 weeks with Primary Care or PT.

### Eligible Participant and Plan Sponsor Pay Contracted Rate

The delay for scheduled appointments, time off from work, personal time coordination, etc. all add to the cost of care.

Many network providers require a direct referral from a primary care provider to PT, increasing the cost of care.

With these delays the participant's MSK pain and discomfort may lessen, reducing the need for immediate care.

Appointment cancelations can be costly, and the minor issue may over time become a more serious chronic MSK care problem.

If the participant is a no show (often with early MSK onset) is still charged co-pay.

Primary Care Provider (NIH/CDC most recent statistics inflating over 7.5%).

The primary care provider has become a hub of referral in the USA healthcare system, with limited knowledge of MSK issues relative to most Physical Therapist (PT).

Are now placed on stringent corporate manifested time allotments per patient.

May be a delay to getting needed care.

Generates additional cost to plan and participant.

### Market Overview

The fastest inflating component in healthcare cost over a 10-year period in the United States based on NIH/CDC data (which is over 5-years old), is the delivery of care at the Primary Care Provider (Dr.), inflating at over 7% per annum delivering symptomatic care to patients.¹ ¹ www.chcf.org/wp-content/uploads/2019/05/HealthCareCostsAlmanac2019.pdf

The same statistical data indicates other U.S. healthcare component cost inflation as follows:

| | |
|---|---|
| Endocrine System Diseases | 6.0% |
| Musculoskeletal System Diseases | 5.5% |
| Respiratory System Diseases | 3.3% |
| Circulatory System Diseases | 2.2% |

Care providers recognize that a healthier Musculoskeletal (MSK) System can mitigate, if not negate, Endocrine (diabetes), Circulatory (heart disease), and Respiratory (breathing) Symptom diseases.

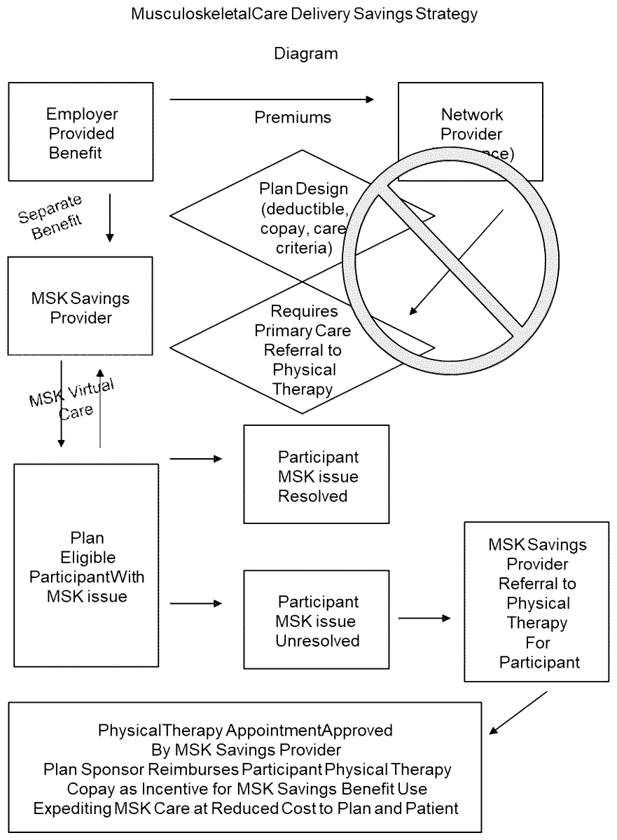

### FIG. 1

a. Chart of Various Major Human Health Diseases (pathologies) in Age Groups.² Illustrates the impact of various pathologies on humans as they age. ² www.boneandjointburden.org

Note: MSK care is the most prevalent in working aged humans.

### FIG. 2

Number of Workdays Lost Based on Strains and Sprains³ Illustrates the number of days missed by individuals in the workforce and the impact on employers from the participant from fairly pathologies. ³ Source for FIGS. 2-6 U.S. Federal Government Center for Disease Control 2013.

FIG. 3 Number of Workdays Lost Based on Traumatic Injury to Soft Tissues.

Illustrates the number of days missed by individuals in the workforce and the impact on employers from the participant what may be fairly normal pathologies.

Note: The almost exact match of the for missed workdays which seems counterintuitive, relative what one would expect.

### FIG. 4

Percentage of Injuries Resulting in Missed Workdays. Illustrates workforce musculoskeletal trauma causes needing participant missing workdays.

### FIG. 5

Number of MSK Cases Resulting in Missed Workdays per Annum. Illustrates individual age groupings relating to workdays missed. Note: Prior to age 25 and after retirement age of 65, the number of missed workdays if much lower.

### FIG. 6

Percentage of MSK Injuries by Body Part. Illustrates the percentage of workforce musculoskeletal care by major body parts. Note: Back (Spine) being the most prevalent. Spinal injury orthopedic remediation surgeries are inherently risky and rarely deliver patent desired outcomes.

In fact, most of the fore-mentioned pathologies often manifest their greatest costs and patient care later in life post-employment, plan sponsor eligibility, and retirement. While MSK issues occur through all life stages from either:
Acute Injury Related MSK pathologies
   OR
Chronic Developing over time often after the age of 35-years old.

With research going back decades from well recognized government and independent research, domestic and abroad studies of ergonomic health in the workforce have long been recognized.

More than half of the American population suffers from back, shoulder, knee, and other joint pain. In total, MSK represents one sixth of all spending in the US healthcare market and is the top cost driver of healthcare spending.

To put this in perspective, the annual cost of MSK conditions (including healthcare costs and lost worker productivity) is around $600 billion, greater than the annual costs of heart disease ($309 billion), cancer ($243 billion), and diabetes ($188 billion).⁴ ⁴ Gaskin D J et al. The Economic Costs of Pain in the United States. Relieving Pain in America: A Blueprint. National Academies Press. 2011.

Musculoskeletal care delivery costs containment is of concern to all parties involved with healthcare delivery, patients, plan sponsors, providers, and insurers all wish to control MSK costs. The current system of MSK delivery is contributing to the inflating MSK delivery costs conundrum in the U.S.A.

All humans benefit from learning from birth to death.

Why restrict access to MSK pathology simple meaningful therapies?

There are multiple therapies, that if educated to utilize, humans can use to improve MSK pain and discomfort. Unfortunately, the medical industrial complex has become addicted to the current MSK delivery strategy, mainlining money.

There are at least 4 to 5 therapies for every muscle/joint group that when provided to a patient can alleviate this pain and discomfort, stimulating oxygen flow to the area and providing relief.

While internet accessibility has opened many to these tools the issue becomes—what if the therapy does not work? Then back into the same MSK cost continuum, more delay, etc.

While some MSK patients with MSK problems have direct access to MSK healthcare specialists through family expertise and knowledge as a PT, massage therapist, or Orthopedic to provide immediate examination or therapy remediation.

The vast majority of MSK patients are not so lucky.

Hence, the MIC scheme penalizes most MSK patients by cloaking basic MSK self-remedial therapies known to these few MSK patients.

Personal MSK discomfort care and knowledge of simple therapies will benefit the MCI participants. Yet, abuses have led observant plan sponsors and network provides to constrain PT interactions, as physical therapy facilities have care delivery equipment replicating workout gyms and staff trainers. To control these abuses plan design implemented the primary care referral requirement to PT.

Covid-19 changed society and healthcare delivery as government mandated personal isolations, replaced Dr. and PT visits.

Staffing reductions occurred as physical therapist, relying on personal interactions with patient to determine pathologies and therapy success, saw an in-ordinate exodus of providers. With reduced staffing in MSK healthcare delivery, being longer-term in pathology development, not being of an immediate concern to care providers post-Covid, has placed additional strain on MSK healthcare delivery.

Regardless, the MCI penalizes MSK eligible participants seeking MSK healthcare delivery to address their MSK concerns, adding participant cost burden either with or without interactions of Dr. for referral to PT delivered MSK input.

Today the medical industrial complex (MIC) scheme is taking advantage of MSK patient pathologies.

MIC is using patient's MSK pathologies to enrich themselves.

Primary provider plan sponsor reimbursements.

Patient copays.

MIC controlling access to basic MSK care.

Requiring primary care visits to the most knowledgeable MSK care specialist, Physical Therapists.

Denying eligible participant reimbursement for PT care if self-referred.

Delaying MSK patient care as primary network providers and facilities appointment access has increased too often over two weeks.

Too often early onset of participant MSK pathologies are ignored, leading to more severe MSK chronic issues requiring Orthopedic care.

Non-Provisional Utility Application Inventor of MCS has experience delivering healthcare services specifically related to musculoskeletal (MSK) system diseases.

MSK pathologies are prevalent in most human beings. Making MSK issues widely discussed within all populations (unlike, herpes or mental health or addition.

With MSK prevalence in the population everyone becomes an expert, has a resolution (misguided or not), experiences empathy (from friend, family, even the HIPAA workforce associate) for the patient's pathology.

EP “Knows” that a multi-million-dollar professional athlete is “cured’ by getting a MRI, (‘didn't player X just return to the field after the MRI? I heard the announcer say so!’).

50% of the US population has experienced MSK pain and discomfort at some point. Unlike other healthcare chronic conditions (i.e., Diabetes, Obesity, etc.), MSK conditions are so common that patients' specific complaints are often openly discussed as almost everyone has or will experience a MSK related problem.

With familiarity of MSK pain and experience from patients comes the potential for abuse within the MSK delivery scheme of care.

In the United States MSK healthcare delivery system patients tend to believe:

Interactions with healthcare provides provide immediate and lasting solutions for patient MSK health through—
Primary Care Providers (Dr.) interactions.

The medical industrial complex has created a care delivery model were Dr. (a General Practitioner. Physician's assistant) acts as a referral hub to physical therapy and orthopedic care with limited MSK pathology knowledge. Even knowledgeable Dr. with the extensive MSK expertise will quickly refer their patient to specialist.

That a Dr., while typically intelligent people, are inflatable and will deliver the black bag “cure” for the patients' MSK pathology.

Dr. will tend to refer to physical therapy (PT) adding additional costs through provider reimbursements by PS and copays by EP.

Pain medications—gateway opioid drug addiction that only masks pain not resolving the pathology.

Provide MSK pathology solutions to patients without patient's active participation in the process—magic wand.

MRI/CT-scans (etc.) deliver relief post procedure.

Diagnostic tools like imaging reveal need for interventions like injections, and/or surgeries for pathologies needing or not needing MSK orthopedic specialist skilled care. Hearing a professional athlete received an MRI and returned to the field has patients confused and demanding that frustrates MSK care providers universally.

Well documented research has shown that MSK healthcare has:

Led to use of Dr. prescribed pain medication (opioid addiction gateway) Wasteful imaging, diagnostics, injections etc. are often patient requested by in-network providers while delivering nominal benefit and generating delay or frustration to patient.

Generated wasted resource allocation by skilled orthopedic surgeons and staff addressing patient MSK pains but not needing surgical remediations.

Current plan design schemes (PD), provided by plan sponsors (PS) and/or network providers (NP), stipulates (requires) that eligible participants (EP) with MSK pain and discomfort must have an appointment with primary care provider (Dr.) for referral to physical therapy/physical therapist (PT) or eligible participants (EP) must bear the total cost of a PT visit directly without the fore referenced Dr. referral.

Note: Typical PT interactions with EP lead to an average of 6 visits in a year.

While this requirement was designed to reduce costs EP care delivery costs to plan sponsors (PS) from abuses by EP self-referring to PT. The requirement for a Dr. visit generates additional costs to the PS and EP with MSK pathologies.

Current design schemes have created a system that delays treatment (appointment) and generates additional costs to both PS and EP. through Dr. reimbursement claims paid by PS and EP copays.

In many ways the current plan design schemes (PD) are disincentivizing EP's MSK health—another cost, time off, why seek help the pain is part of aging—all true, but not contributing to healthcare cost control and EP pain free living.

With MSK commonality humans have assumed the inevitability that MSK pain and discomfort is going to happen and there is nothing to do about it. Too often participants in healthcare plans either ignore the rare MSK discomfort or wait until the MSK issues becomes debilitating.

The influence of primary care provider (Dr.), network providers (NP—i.e., health insurance companies) and plan sponsors (PS—i.e., multi-employer trust, employers) have built healthcare delivery schemes of healthcare that have created a mess for the average consumer.

Changing the built in scheme of the United States healthcare delivery of MSK is never going to be easy.

Musculoskeletal (MSK) care in particular is forth with abuse.

As delays in any disease's remediation may lead to completely unnecessary and expensive care resolutions for patients, costs borne by the plan sponsors (PS), network providers (NP) and eligible participants (EP) alike (i.e., extend cough in patient may be allergy or cancer), if ignored, one patient's care costs the whole.

So, the little cough becomes cancer—knee pain becomes knee replacement.

How do consumers, providers, insurers promote and incentive EP to take an active role in their personal MSK health to improve their lives?

Not knowing better, eligible participants (EP) of healthcare benefit plan design (PD) are forced to spend unnecessary time, effort, dollars, delay of care and resolution for their MSK pathologies specific to the EP's need.

MCS is designed to address these fore-mentioned issues for all related parties of the MSK care delivery in the United States going forward.

Current PD requirements of requiring primary care provider (Dr.—General Practitioner, Physician's Assistant) appointment for a physical therapist (PT) referral are built into PDs generating the opportunity for MCS strategy.

26 states in the United States allow citizens to self-refer to PT. HOWEVER, if the patient of PT does not follow current scheme protocol of Dr. referral, EP may pay all associated cost of PT visits directly, without PD coverage negotiated contracted benefits.

It is recognized that EP abuses of self-referral to PT, prior to the current PD scheme of Dr appointment for referral, have been historically problematic. Necessitating the PD design.

MCS is aligned to address this pitfall.

MSK pathologies are the most common disease in all humans particularly as the aging process occurs, most typically after the age of 35.

MSK pathology (MP) may be related to acute (injury related) or chronic (over time to aging/repetitive motion/arthritic) pain and discomfort in humans. Rarely would human beings not experience personally or through known persons to them the effects of MP.

MCS strategy implementation delivers remediation to NP, PS, EP and will reduce waste of current MSK care.

MCS reduces tangible and intangible costs from current protocols of MSK healthcare benefit scheme is which include:

Dr. appointment delays, PS contracted reimbursements.

EP copayments, work hour loss, inconvenience, MCS further incentives EP by having in-network referred PT visit(s) copay(s) waived post MSS interactions by PS.

With delivery of MSK therapies unknown by MCS provider to participants, without the Dr. associated billing costs, including PS reimbursements and MSK patient. The EP provided meaningful and supportive concepts for personal MSK health improvement and would retain the knowledge into the future and apply and/or share.

The Utility patent design care clinicians may refer MKS patients to in-network PT directly without current networks/plan sponsors scheme requiring a Dr. visit for PT referral, generating savings for plan sponsors (PS) and MSK patients (P) alike and reducing EP pathology care delays from Dr. appointment for PT referral, if needed.

Musculoskeletal Care Deliver Saving Strategy delivers inexpensive patient centric MSK care that provides EP a pathway of supported understanding and guidance for a better life.

Through the Non-Provisional Utility patent research there has been no discovery of conflicting patents.

### SUMMARY

### Musculoskeletal Care Delivery Savings Strategy (MCS)

Musculoskeletal Care Delivery Saving Strategy (MCS) utility patent design alters the current Musculoskeletal (MSK) healthcare delivery scheme design strategy provided by network provider (NP) and plan sponsors (PS) via the plan design (PD) benefit provided eligible participants (EP).

MCS removes the criteria of primary care provider (DR) visits for referral to physical therapy (PT). MCS design provides savings to PS and EP alike.

Current healthcare delivery for musculoskeletal care in the United States plan design (PD) requires a primary care provider (DR) appointment to discuss eligible participant (EP) musculoskeletal pathology.

DR has become a hub of medical care for EP. Managing pharmaceuticals, basic symptoms, etc. for patients. While MSK issues may be acute or chronic at any time of the human life cycle, chronic MSK issues manifestations tend to increase with aging. The DR are less familiar with younger EP as a population as individuals, save standard health and/or reproductive examinations. As the EP ages the familiarity of a DR with the EP improves with increased pathologies requiring DR observation, therapies, and management. MSK increases significantly with age.

Network providers (NP) insurance providers or PS contract with DR to provide medical services to EP. DR compensation from all EP consultation appointments are reimbursed as contracted with NP/PS and include EP copay.

Several decades ago, the Internal Revenue Service removed athletic club memberships, as a deductible expense. Shortly after NP and PS realized that Physical Therapy (PT) were seeing increase claims by EP. With the need for PT to have facilities with equipment and personnel similar to athletic club facilities.

The requirement of DR interaction with EP, regarding MSK pathologies for referral to PT, placed protection from this PT abuse scheme by EP.

Today this requirement is wasteful, delays MSK care, has placed the EP at the vagaries of the medical industrial complex with double copay to access PT WHEN needed, costs PS double reimbursements to DR and PT.
a. MCS reduces the need for redundant costs.
b. MCS provides EP quicker access to MSK pathology consultation.
c. MCS provides PT referral post consultation.
d. MCS increases MSK self-remediation therapies awareness to EP.
e. MCS reduces wasted interaction with DR and EP.
f. MCS provides EP access to PT with knowledge of MSK concern.
g. MCS Post PT consult, EP needed orthopedic care and/or surgeries are expedited.

Musculoskeletal Care Deliver Savings Strategy is designed to introduce patients to better understanding of musculoskeletal heath through education, interaction, and restoration, saving plan sponsor, and participant from the waste of MSK care prevalent in the United States.

### DETAILED DESCRIPTION

### Musculoskeletal Care Delivery Savings Strategy (MCS)

Non-provisional Utility patent (NUP) design alters the current Musculoskeletal (MSK) healthcare delivery scheme design (PD) strategy provided by network provider (NP) and plan sponsors (PS).

NP/PS may or may not alter PD for MCS benefit.

Musculoskeletal Care Delivery Savings Strategy (MCS) is provided to eligible participants (EP) as PS benefit for MSK care delivery option.

EP is allowed to continue to follow current PD protocols as designed, with affiliate copays and delays, for PT consultations, MCS is contacted by EP for MSK consultation.

MCS is benefit alternative to arranging a primary care provider (PC) appointment as required in current NP/PS PD scheme.

EP is incentivized to interact with MCS rather than PC regarding EP musculoskeletal pathology concern (MPC).
a. Saving both EP and PS, tangible and intangible costs:
   i. Time.
   ii. Delayed care.
   iii. Workforce participation loss.
   iv. EP Copay.
   v. PC Reimbursement

EP interacts with MCS provider staff (MP).

MP interview EP (documented).
a. EP health statistical information.
b. Discussing EP specific MPC.
c. MPC timeline (acute/chronic) determined.

MCS provides EP self-remedial therapies (education) via EP desired means of delivery.
a. Text
b. Email
c. Other

MP and EP schedule follow up appointment (optional).

EP learns self-remedial MSK care unknown to EP.

MPC improves.

MCS benefit delivers improved MPC to EP.
a. Win—Win—Win

EP realizes No improvement of MCP from MP interaction/s.

MCS as plan benefit is authorized to provide physical therapy (PT) referral to EP as required by PD.

EP has PT appointment.
a. EP copay is reimbursed as incentive for MCS MP interaction/s.

EP appointment with PT.
a. Appointment to network PT (subject to PD provider constraints).
b. PT skilled diagnostics of MPC to EP provided.
   i. PT are not licensed to write scripts for:
      1. Injections
      2. Imaging diagnostics.
      3. Medications.
c. Can determine from physical examination EP MPC severity.

PT interaction/s with EP communicated to MCS MP for documentation.

Should PT consult with determine need for orthopedic specialist care (DO) directed referral can be made from MCS and MP to EP network provider.

MP and EP working with DO discuss MSK restoration for EP MPC and support to achieve best practice and outcome for EP.

Through application of MUSCULOSKELETAL CARE DELIVERY SAVINGS STRATEGY, the following benefits are provided as follows:

### Eligible Participant

a. Less costs.
   i. Copay.
   ii. Lost hours.
b. Reduced appointment scheduling time.
   i. Interaction with is MP virtual on EP schedule.
   ii. No primary care appointment.
   iii. MCS MP referral to PT delivered quicker.
c. Better understanding of MSK care

### Plan Sponsor/Employer

a. MSK related work loss decreases.
b. No direct reimbursement to primary care provider from removed referral requirement.
c. Increased MSK health awareness.
d. Eligible participant copay reimbursement incentives.
   i. Save MSK healthcare costs under current scheme.
   ii. Encourages quicker interaction with MSK specialists.
e. Improved participant MSK.
   i. Reducing long-term MSK surgical therapy interventions.
   ii. Moves major MSK cost components of population to post retirement care (Medicare).

The MCS may be offered as a required or voluntary benefit to EP.

If required by PS, a PD change may be necessary.

Optimally, the MCS is offered as a voluntary benefit to EP.

MCS provides EP focused MSK care relief, only.

MCS's benefit is MSK care relief provided directly to EP relating to MPC specific care delivery as follows:
EP expedited care.
Controlled PT referral.
Reduced reimbursement to Dr. by PS.
Reduced copayments to in-network provider, Dr./PT, from EP.

MCS may be HIPAA compliant and offered either within or outside the PS Dr. network.

MCS may or may not provide medical advice to EP depending on the structure of NP/PS/PD depending on the PS and NP constraints and needs.

The MCS will disrupt the current transitory system of MSK care in the United States healthcare delivery system generating significant cost savings for MSK care going forward with better outcomes for EP, PS, and Dr. alike.
a. CP provides specific therapies to alleviate MSK patient's pathology via MSK patients' desired means of delivery.
b. CP follows up as arranged with MSK patients prior to initial interaction closure.
c. MSK patients utilize self-remedial tools.

Provided necessary relief, CP and MSK patients have exit interviews documenting MSK care. Musculoskeletal Care Delivery Saving Strategy patent will radically change the current MSK care paradigm of waste, delay, medical industrial complex costs schemes, and empower patients with patient centric MSK care knowledge and therapies. Human beings understanding their personal body's MSK system aches and pains, needs, cares, and management benefit their own health as poor MSK health often leads to poor general health.

1. Musculoskeletal care delivery will benefit from utility design.

2. Reduces cost of care to plan sponsors and eligible participants.

3. Provides participants with therapeutic knowledge specific to their MSK need.

4. Incentives participants, through plan sponsor provided reimbursement of copay for physical therapy visits.

5. Removes required primary care appointment to receive physical therapy referral.

6. MSK care delivery using a self-centric focused participant care.

7. Removes MSK care strategy obstacles that deter participants from seeking MSK care.

8. Negates wasteful appointments to Primary Care and Orthopedic physicians for physical therapy.

9. Provides support and guidance for participants requiring additional MSK pathology care at reduced cost.

10. Saves time and money to plan sponsor and plan participant.

11. Removed primary care requirement reduces total MSK spend that over time will impact participant care as poor MSK health may lead to other pathologies and addictions.

12. Attacks the core of the current the medical industrial complex money-making scheme waste and delay.

13. Physical Therapist are more knowledgeable about MSK System than Primary Care professional but are not allowed to write prescriptions for medications, x-rays, other imaging, surgeries, or therapies.

14. The PT can through examination determine if the patient has an acute or chronic MSK issue, needs a referral to an Orthopedic provider or needs other non-invasive therapies.

15. Faster patient MSK care response for MSK care, referral need resolution for PT.

16. No Participant Copay or time off work for provider office visit for initial screening.

17. Unique to each plan sponsor and network provider.

18. More convenient care for participants with MSK skilled assistance.

19. Up to 80% of MSK issues can be resolved outside traditional model.

20. Generates savings to participant and sponsor as early MSK onset can often be resolved with MSK education and self-remediation.

21. Musculoskeletal Care Delivery Saving Strategy patent will change the current MSK care paradigm of waste, delay, medical complex costs schemes, and empower patients with patient centric MSK care knowledge and therapies as an active participant in their own MSK system's health.
